(19) United States
(12) Patent Application Publication
(10) Pub. No.: US 2026/0098870 A1
(43) Pub. Date: Apr. 9, 2026

(54) CIS-TRANS ISOMERIZATION OF OXYTOCIN

(71) Applicant: Joel Steven Goldberg, Hillsborough, NC (US)

(72) Inventor: Joel Steven Goldberg, Hillsborough, NC (US)

(21) Appl. No.: 19/415,487

(22) Filed: Dec. 10, 2025

(51) Int. Cl. G01N 33/74; G01N 21/35; G01N 24/08; G01N 27/623; G01N 30/02

(52) U.S. Cl. CPC G01N 33/74; G01N 21/35; G01N 24/087; G01N 27/623; G01N 30/02; G01N 2030/027; G01N 2333/575

(57) ABSTRACT

Failures in establishing trust during international conflict negotiations frequently result in warfare, as ideological differences often cannot be resolved through rational discourse alone. Empathy between opposing parties is essential for building trust, and oxytocin is recognized for its critical role in facilitating empathy, trust, inter-brain synchrony, and cooperative decision-making. However, prevailing scientific studies typically do not differentiate between the cis and trans isomers of oxytocin, despite evidence that only the trans isomer is pharmacologically active. The present invention provides methods for the separation and quantification of oxytocin isomers, with particular emphasis on the trans isomer's function in social synchrony and decision-making within conflict negotiation contexts. Additionally, the invention explores the role of extremely low frequency electromagnetic radiation permeating the human central nervous system interconnecting neurochemical changes among groups. These advances clarify the unique neuropeptide functions of oxytocin and enhance understanding of its impact on cooperation and conflict resolution.

### CROSS-REFERENCE TO RELATED APPLICATIONS

This application claims the benefit of U.S. application Ser. Nos. 19/191,666, 18/797,623, 17/958,328, 16/706,760, 16/139,062, 15/191,673, 15/057,146, 14/995,376 and 14/844,202 which are incorporated by reference.

### FEDERALLY SPONSORED RESEARCH AND DEVELOPMENT

Not applicable

### BACKGROUND OF THE INVENTION

### Failed Negotiations

The 21st century has been marked by widespread violence, with major conflicts such as Ukraine-Russia, Israel-Gaza (Hamas), Israel-Lebanon (Hezbollah), Israel-Iran, the civil war in Yemen, and the ongoing crisis in Syria. A recurring theme among these wars is the breakdown of negotiations before violence erupts; these disputes are rarely sudden, but rather stem from long-standing, unresolved issues. Another contributing factor is the reluctance of political leaders to invest in proactive measures to address these problems, often allowing tensions to simmer until they reach a critical point. For example, the current dispute over Taiwan's sovereignty versus its potential assimilation into China is a situation where substantial resources should be devoted to resolution, rather than waiting for conflict to escalate.

Many leaders focus on reacting to crises instead of tackling their root causes. Their primary concern is often the protection or expansion of their nation's interests, sometimes resorting to force if necessary. Notably, Ronald Reagan once suggested that contact with a superior alien civilization could instantly shift the priorities of world leaders. Similarly, in 1947, Albert Einstein addressed the United Nations, advocating for a supranational authority with the power to prevent wars.

The greatest obstacle to resolving international conflicts is a lack of trust. Trust is fundamental to the functioning of human society, but when opposing ideologies are involved, rational arguments alone may not be enough to build it. In these cases, empathy can help bridge ideological divides and foster trust. While there is debate about cooperation within and between groups, many sources suggest that empathy and trust are linked to the presence of trans oxytocin in the central nervous system (CNS). Research indicates that trans oxytocin in the CNS is associated with inter-brain synchrony, which promotes cooperative rather than competitive decision-making. Except for possible effects from vasopressin, this role of trans oxytocin appears to be unique among known neuropeptides and neurotransmitters.

### General Considerations of Oxytocin

Oxytocin is a cyclic nonapeptide neurotransmitter stored in the posterior pituitary, known for its roles in empathy, altruism, love, trust, and interbrain synchrony. Structurally, oxytocin exists in both cis and trans isomeric forms due to a pseudo double bond between a proline residue and an adjacent cyclic disulfide ring. Research indicates that the trans isomer is the primary agonist that binds to the oxytocin receptor. However, most scientific literature discussing oxytocin's behavioral, pharmacokinetic, and pharmacodynamic effects does not distinguish between these isomers, raising questions about the accuracy of such findings. Additionally, oxytocin is prone to degradation during storage.

While oxytocin is linked to cooperation within groups, it may paradoxically contribute to intergroup non-cooperation, xenophobia, and aggression. The cis and trans forms of oxytocin absorb different wavelengths of electromagnetic radiation (EMR), and a dynamic equilibrium exists within the open thermodynamic system of the CNS. Differences in free energy (ΔG) between the isomers may provide information interconnecting extremely low frequency (ELF) EMR, which permeates the CNS potentially explaining the synchrony observed in hyperscanning experiments and in nature, a phenomenon often occurring without physical contact.

Without the ability to separate and measure each oxytocin isomer in biological samples, the validity of past and future scientific studies remains incomplete. The aim of this invention is to introduce methods for separating and quantifying oxytocin isomers, which may provide deeper insight into interbrain synchrony and cooperative decision-making. Ultimately, this work seeks to encourage greater investment in the interdisciplinary fields of neuroscience and conflict negotiation.

### PRIOR ART

### Non-Patent Literature

Fuller, D. R., et al. “Cis→Trans Isomerization of Pro(7) in Oxytocin Regulates Zn(2+) Binding.” Journal of the American Society for Mass Spectrometry, vol. 27, no. 8, 2016, pp. 1376-1382.

Ren, H. C., et al. “Revealing the Relationship between Electric Fields and the Conformation of Oxytocin Using Quasi-Static Amide-I Two-Dimensional Infrared Spectra.” ACS Omega, vol. 7, no. 4, 2022, pp. 3758-3767.

Mu, Y., et al. “Oxytocin Enhances Inter-Brain Synchrony During Social Coordination in Male Adults.” Social Cognitive and Affective Neuroscience, vol. 11, no. 12, 2016, pp. 1882-1893.

Tabak, B. A., et al. “Advances in Human Oxytocin Measurement: Challenges and Proposed Solutions.” Molecular Psychiatry, vol. 28, no. 1, 2023, pp. 127-140.

Hruby, V. J., et al. “NMR Studies on the Conformation of Derivatives of the Side Chain of Oxytocin: Examples of Cis-Trans Isomerism.” Proceedings of the National Academy of Sciences USA, vol. 68, no. 2, 1971, pp. 450-453.

Larive, C. K., Guerra, L., Rabenstein, D. L. “Cis/Trans Conformational Equilibrium Across the Cysteine6-Proline Peptide Bond of Oxytocin, Arginine Vasopressin and Lysine Vasopressin.” Journal of the American Chemical Society, vol. 114, 1992, pp. 7331-7337.

Larive, C. K., Rabenstein, D. L. “Characterization of the Dynamics of the Cis-Trans Isomerization About the Proline Amide Bond of Oxytocin and Arginine Vasopressin in Aqueous and Methanol Solutions.” Journal of the American Chemical Society, vol. 115, 1993, pp. 2833-2836.

Grifnee, E., et al. “Development and Validation of a Highly-Sensitive, Quantitative LC-MS/MS Assay to Evaluate Plasma Oxytocin.” Journal of Mass Spectrometry Advances in Clinical Lab, vol. 36, 2025, pp. 19-28.

### Patent Literature

CN106831951A, “A Kind of Purification Process of Oxytocin,” published Jun. 13, 2017.

CN101235081B, “Method for Preparing Oxytocin,” published Jul. 7, 2010.

CN108721598B, “Preparation Method of Oxytocin in Raw Material, Pharmaceutical Composition and Preparation Thereof,” published Jun. 5, 2020.

WO2019032946A1, “Method of Manufacturing of Oxytocin,” published Feb. 14, 2019.

WO2022181717A1, “Oxytocin Purifying Method and Measuring Method and Kit,”published Sep. 1, 2022.

JP7690017B2, “Oxytocin Purification Method, Measurement Method and Kit,”published Jun. 9, 2025.

WO2016140063A1, “Method for Measuring Oxytocin with High Sensitivity,”published Sep. 9, 2016.

WO2024044633, “Oxytocin Derivatives and Methods of Use,” published Feb. 29, 2024.

WO2025146648, “Oxytocin Compositions and Methods,” published Jul. 10, 2025.

WO2019169342A1, “Labeled Oxytocin and Method of Manufacture and Use,”published Sep. 6, 2019.

US20240288452A1, “Detection of Oxytocin in a Biologic Sample,” published Aug. 29, 2024.

US20160377635A1, “Measurement of Oxytocin and Vasopressin,” published Dec. 29, 2016.

EP2161030A1, “Oxytocin Formulations and Uses Thereof,” published Mar. 10, 2010.

EP2326341B1, “Oxytocin Formulations and Uses Thereof,” published Jul. 4, 2012.

### SUMMARY OF THE INVENTION

Efforts to resolve international conflicts through negotiation frequently fall short, often leading to violence as the default outcome. When disputes are rooted in deeply held ideologies, rational discussion alone may not be enough to build the trust necessary for peaceful resolution.

Recent research suggests that higher levels of the trans isomer of oxytocin in the CNS could promote empathy and trust among negotiators. However, most studies on oxytocin's influence on human behavior overlook the distinction between its cis and trans forms. This is significant because the trans isomer is believed to be responsible for oxytocin's effects on empathy, trust, and the synchrony observed between brains during cooperative tasks. Such synchrony, documented in hyperscanning experiments, occurs even without physical contact, implying that information is transmitted either through responsiveness to shared stimuli or directly between individuals.

This phenomenon is reminiscent of Huygens'pendulum clocks, which synchronized their movements through a physical connection (wood beam), a principle that may also apply to biological systems, where ELF EMR could interconnect the oscillations of oxytocin isomers. These oscillations are associated with differences in ΔG that could interconnect energy information to ELF EMR as modulations in amplitude, frequency, or phase like radio communications.

Each oxytocin isomer absorbs EMR at different wavelengths, and these interactions may play a role in synchrony. The relationship between the ΔG of the cis trans isomerization and the energy densities from the EMR can be expressed as:

U=U_{O}+∫[½ε_{O}|E|²+(½μ_{O})|B|²]dV

where:
a. U is the total energy,
b. U_{O} is the classical thermodynamic energy, which includes the Gibbs free energy change (ΔG),
c. ε_{O} is the permittivity of free space,
d. E is the electric field strength,
e. μ_{O} is the permeability of free space,
f. B is the magnetic field strength,
g. dV is the differential volume element.

The term ∫[½ε_{O}|E|²+(½μ_{O})|B|²]dV represents the energy contribution from the EMR within the specified volume. In the CNS, ε and μ differ than in free space.

Although enzymes like peptidyl proline isomerase (PPIase) can lower the energy required for cis-trans isomerization, it is unlikely that non-thermal, non-ionizing EMR can directly alter this equilibrium in living organisms. Laboratory studies estimate that converting cis oxytocin to its trans form requires about 20 kcal/mol, but in the body, this energy barrier may be reduced by factors such as enzyme activity, zinc ions, hydrogen bonding within the molecule's cyclic disulfide ring, temperature, and pH. Even with these reductions in activation energy, ELF EMR found in nature probably do not have the energy from photons or electromagnetic fields to break the pseudo double bond in oxytocin to produce cis-trans isomerization. However small shifts in the balance toward trans oxytocin could have significant effects on behavior, given the CNS's complex and dynamic, often chaotic, nature. Developing reliable methods to separate and measure oxytocin isomers will deepen our understanding of this important neurotransmitter. Despite tens of thousands of research papers on oxytocin, few specify which isomer is present. Beyond its relevance to conflict negotiation, trans oxytocin may be crucial for social synchrony, highlighting the need for greater investment in research at the intersection of neuroscience and negotiation science.

### DETAILED DESCRIPTION OF THE INVENTION

This invention is founded on the following principles:

1. The CNS operates as an open thermodynamic system, generating ELF EMR and exhibiting stochastic, periodic, and chaotic behaviors.

2. On a non-quantum level, ions move rapidly through CNS channels, producing ELF EMR that may be detected as electroencephalogram (EEG) and magnetoencephalogram (MEG) signals.

3. Non-thermal, non-ionizing ELF EMR permeates the CNS and interconnect information relevant to inter-brain or brain-to-stimulus synchrony.

4. Synchrony observed in hyperscanning experiments and in nature often occurs without physical contact, suggesting that either sound, light, or ELF EMR may facilitate this interconnection.

5. No published hyperscanning experiments have been found where subjects are placed inside Faraday cages specifically to test the role of ELF EMR in inter-brain synchrony.

6. Synchrony requires that individuals receive, process, and respond to shared cues and without a mechanism for information exchange, coordinated neural or behavioral activity would not occur except by coincidence.

7. Within the human CNS, oxytocin exists in a dynamic equilibrium between cis and trans isomers which is a distinction rarely addressed in human studies.

8. The trans isomer of oxytocin is closely linked to empathy, trust, altruism, and inter-brain synchrony.

9. Ambient non-thermal, non-ionizing ELF EMR, whether from photons or energy densities, does not provide enough energy to alter the cis-trans equilibrium of oxytocin, though ELF EMR can still interconnect information about the equilibrium within the CNS.

10. The cis-trans isomerization of oxytocin is governed by both kinetic and thermodynamic factors.

11. The rate of cis-trans isomerization is primarily regulated by PPIase and can be modeled using the Arrhenius equation.

12. Thermodynamic control of the cis-trans equilibrium is determined by the ΔG between the isomers.

13. The interaction between ELF EMR and the chemistry of the human CNS can be described through the energy density equation:

U=U_{O}+∫[½ε_{O}|E|²+(½μ_{O}) |B|²]dV

14. Cooperative decision-making is associated with CNS synchrony and elevated levels of trans oxytocin.

15. Separating and measuring trans oxytocin and the ratio of cis to trans isomers will provide deeper insight into its pharmacodynamics and help validate or challenge existing research findings.

### Structure and Function of the CNS

At the classical (non-quantum) level, the CNS operates as a complex chemical-electrical system. Chemical reactions within the CNS generate electrical signals, while the movement of ions through neural channels produces action potentials and both excitatory and inhibitory postsynaptic potentials. The rapid transit of ions through these channels results in time-varying electric and magnetic fields, which in turn generate endogenous ELF EMR detectable EEG and MEG signals.

When external ELF EMR permeates the CNS, it can interact with these internally generated fields, thereby influencing neural activity and chemical equilibria. This dynamic exchange of energy demonstrates that both endogenous and exogenous ELF EMR contribute to the electromagnetic environment of the CNS. Such interactions provide a physical basis for the modulation of neurochemical processes and the phenomenon of inter-brain synchrony.

ELF EMR produced by the CNS can store and transmit information.

Current scientific understanding suggests that consciousness is dependent on the CNS, and some theoretical models propose that consciousness may be encoded within the ELF EMR generated by neural activity. Observations of altered states including general anesthesia, meditation, coma, and cerebral ischemia support the hypothesis that consciousness is closely linked to CNS-generated ELF EMR. Furthermore, psychopharmacological research indicates that changes in neurotransmitter levels affect ion movement, thereby influencing cognitive processes and behavior.

### Human Behavior and the Motor System

Human behavior is fundamentally connected to the motor system. Without the capacity to speak, write, gesture, or move, behavior is essentially absent except in rare cases of automatic bodily responses. When considering human behavior, it's important to recognize that voluntary motor actions result from the integration of information from various regions of the brain. Whatever internal processes occur within the mind, the final expression of behavior is a motor output. As a result, the coordination and synchrony of behavior directly reflect the synchrony of the motor system.

### Hyperscanning and Human Behavior

Hyperscanning is defined as the concurrent monitoring of the CNS of two or more individuals. Empirical studies utilizing this technique have demonstrated that neural synchrony, characterized by the coordination of brain activity across multiple participants, is strongly associated with cooperative decision-making. Such synchrony is not exclusive to humans; it is also observed in collective behaviors among animal groups, including dolphin pods, fish schools, bird flocks, and insect swarms. In humans, episodes of group synchrony are correlated with enhanced feelings of happiness and social connection, as evidenced during communal activities such as concerts or interpersonal interactions between romantic partners.

The underlying mechanisms responsible for neural synchrony remain incompletely understood. However, it is established that some form of information exchange must occur between oscillating biological systems to facilitate this phenomenon. Historical observations, such as Huygens' synchronization of pendulum clocks via physical coupling, suggest that subtle connections can interconnect coordinated oscillations.

In biological contexts, ELF EMR is proposed as a mediating factor that enables synchrony among living organisms. Notably, hyperscanning experiments reveal that participants can achieve neural synchrony without direct physical interaction. This observation supports the hypothesis that communication may be mediated by external ELF EMR, which has the potential to interconnect motor responses and behavioral outputs across individuals.

### Mechanism of Inter-Brain Synchrony in Hyperscanning

Previous research indicates that ELF EMR may influence CNS chemistry in various ways, including altering oxidation-reduction reactions, Zeeman splitting of radical pairs, methylating DNA, phosphorylation, modifying protein conformations, and impacting the blood-brain barrier.

Laboratory studies have shown that weak magnetic fields (in the micro-Tesla range) can affect oxygen reduction reactions (ORR) through paramagnetic effects, thereby influencing ATP production. Since ORR processes are autocatalytic, their oscillations could be interconnected with ELF EMR, potentially leading to inter-brain synchrony observed in hyperscanning studies.

This invention proposes an alternative mechanism: the interconnection of differences in ΔG of cis-trans oxytocin isomerization with ELF EMR. As the concentrations of oxytocin isomers fluctuate the energy changes could be interconnected to ELF EMR which permeate the CNS, producing synchrony in groups or dyads and possibly explaining the results seen in hyperscanning experiments.

While other proteins also contain proline residues and undergo cis-trans isomerization, the presence of a cyclic disulfide ring adjacent to proline found in oxytocin and vasopressin is unique. This structural feature may lower the activation energy required for these oscillations, making oxytocin particularly susceptible to ELF EMR interconnected synchrony.

### Cis-Trans Isomerization of Oxytocin

The cis-trans isomerization of oxytocin, specifically at the peptide bond between cysteine-6 (Cys6) and proline-7 (Pro7), serves as a model for similar processes in other biological molecules, including enzymes, receptors, and neurotransmitters. This isomerization has been confirmed through in vitro techniques such as NMR, infrared, and mass spectrometry, as well as computational modeling, which help characterize its kinetics, energetics, and structural effects.

Despite its importance, cis-trans isomerization is often overlooked in studies of molecular structure and activity, partly because these transitions occur rapidly and are challenging to measure and separate. Additionally, the lowest energy state does not always correspond to a specific conformation. As a result, many oxytocin measurements and reported outcomes may be unreliable, since they do not distinguish between the cis and trans forms.

The trans form is more compact, with stronger hydrogen bonds and a more stable disulfide bond than the cis form. This motif is rare among CNS enzymes, receptors, or neurotransmitters, with vasopressin being a notable exception. However other cyclic peptides, such as cyclotides, conotoxins, and cysteine-proline-proline-cysteine motif peptides, can also have a cyclic disulfide ring next to proline, which imparts greater stability and distinct structural properties.

Experimental evidence from D2 IR spectroscopy shows that electric fields can induce cis-to-trans isomerization in oxytocin, with a minimum field strength of 1.028 V/nm applied orthogonally. Computational chemistry studies (DFT/B3LYP) have mapped the energy landscape of this process, though in vivo activation energies remain undetermined.

Enzymes such as PPIases can lower the activation energy required for this isomerization, which is important for protein folding and function. Most common neurotransmitters do not undergo cis-trans isomerization, and while some neuropeptides contain proline residues, they typically lack an adjacent cyclic disulfide ring. Certain G protein-coupled receptors (GPCRs) with looped cyclic disulfide rings may also undergo similar isomerization, influencing ion channel activity.

### Cis-Trans Isomerization of Retinal

A key example from human vision is the cis-trans isomerization of retinal. When visible light strikes retinal, it provides enough energy to unpair the two electrons in the cis double bond. This allows the C—C bond to rotate 180 degrees, converting the molecule from its cis form to the trans geometric isomer. This photochemical process requires an activation energy of 40-50 kcal/mol, with a ΔG of −2 to −5 kcal/mol favoring the trans isomer. Remarkably, this transformation occurs extremely rapidly within approximately 2×10⁻¹³ seconds. It is unlikely that visible light can penetrate the sella turcica interacting with the posterior pituitary and oxytocin is not a known chromophore.

### Role of the Disulfide Ring in Oxytocin

The disulfide ring in oxytocin plays a crucial role in its structure and function. By stabilizing the transition state through intramolecular interactions like hydrogen bonding, the ring lowers the energy barrier and accelerates the rate of cis-trans isomerization at the Cys6-Pro7 peptide bond. Its cyclic nature makes oxytocin more stable than linear peptides, protecting it from enzymatic breakdown and increasing its resistance to heat and chemical changes. These conformational dynamics are essential for oxytocin to maintain the precise three-dimensional shape needed for effective receptor binding and biological activity. In summary, the disulfide ring is vital for oxytocin's structural integrity, influences the rate and balance of cis-trans isomerization, and is key to its physiological effects.

### Oxytocin and Interbrain Synchrony

There is ongoing debate about whether oxytocin promotes synchrony between brains, with some studies indicating it enhances synchrony within groups but not between different groups. Oxytocin has been shown to increase inter-brain synchrony during social coordination tasks. This synchrony can be objectively measured using techniques such as EEG, MEG, fMRI, and fNIRS.

Typically, the effect is observed after administering intranasal oxytocin, which influences neural oscillations. For example, research has found that pairs of individuals (dyads) treated with intranasal oxytocin display greater synchrony in alpha-band neural activity during cooperative tasks compared to control tasks, and this increased synchrony is linked to improved interpersonal coordination. However, the way oxytocin is administered and its pharmacokinetics remain controversial, and most studies do not account for the equilibrium between cis and trans forms of oxytocin despite evidence that the trans isomer is the pharmacologically active form.

### Cis-Trans Isomerization of Oxytocin and Zinc Ions

Research has shown that zinc ions can promote the conversion of cis oxytocin to its trans isomer, which is crucial for effective receptor binding. However, these findings are based on in vitro experiments not environments typically present in the CNS, whether in cerebrospinal fluid (CSF) or brain tissue. Many of these studies are conducted at non-physiological pH levels. As a result, while zinc can induce cis-to-trans isomerization under laboratory conditions, its impact under normal biological circumstances remains unclear.

### Oxytocin and Vasopressin: Shared Receptor Dynamics

Oxytocin and vasopressin are structurally similar peptides, differing by amino acid substitutions at the third and eighth positions. Oxytocin contains leucine and isoleucine, while vasopressin has arginine and phenylalanine respectively. Both molecules feature a cyclic disulfide bond adjacent to a proline residue, which allows for cis-trans isomerization. The GPCR for oxytocin can also bind vasopressin, and vice versa, leading to complex physiological interactions within the CNS. This receptor cross-reactivity complicates the understanding of oxytocin's specific roles and effects in neural processes.

### Single Nucleotide Variants of the Oxytocin Receptor

Single nucleotide variants in the oxytocin receptor, such as the rs53576 polymorphism, result from a substitution of guanine (G) with adenine (A). Some research suggests that individuals with the G-G allele may exhibit higher levels of empathy and altruism compared to those with the A-A allele.

Oxytocin is a highly conserved neurotransmitter across the animal kingdom, but there is considerable variation in both its molecular structure and the structure of its GPCR among species. The oxytocin systems in chimpanzees, gorillas, and bonobos are most similar to those in humans, indicating that findings from studies in mice, rats, cats, and dogs may not be directly applicable to humans. Therefore, further research in these animal models may not effectively advance understanding of human oxytocin biology.

### Variability of PPIase Activity and Its Impact on Oxytocin Isomerization in Humans

PPIases exhibit variable activity in humans due to genetic, developmental, physiological, and environmental factors. This variability directly influences the cis-trans equilibrium of oxytocin in the CNS. Elevated PPIase activity accelerates the interconversion between cis and trans oxytocin, favoring the pharmacologically active trans isomer associated with empathy, trust, and inter-brain synchrony. Conversely, reduced PPIase activity slows isomerization, potentially increasing the proportion of cis oxytocin and diminishing the availability of the active trans form.

The estimated first order rate constant of in vivo PPIase catalysis of oxytocin cis-trans isomerization is 1-10 s⁻¹ with a half-life of 0.07-0.7 seconds compared to a rate constant of 0.01-0.1 s⁻¹ with a half-life of 7-70 seconds in aqueous in vitro solutions. PPIase differences may contribute to individual variability in social cognition and behavior and altered PPIase activity in pathological states could disrupt oxytocin's neurochemical and behavioral effects. Therefore, measuring PPIase activity and oxytocin isomer ratios may serve as valuable biomarkers for negotiations, neuropsychiatric conditions, and therapeutic response.

### Hydrogen Bonding Influences Cis-Trans Isomerization of Oxytocin

Hydrogen bonding plays a significant role in stabilizing the structure of oxytocin, particularly through interactions within its cyclic disulfide ring. These intramolecular hydrogen bonds can lower the energy barrier for cis-trans isomerization making the transition between forms more attainable. The pattern and strength of hydrogen bonding differ between the cis and trans isomers, affecting their stability and the equilibrium between them.

The differences in hydrogen bond patterns between cis and trans isomers results in a shift of the amide-I absorption peak in the infrared spectrum and the exact wavenumber and shape of the amide-I band can be used to distinguish between isomer forms. In oxytocin, the trans isomer tends to be more stabilized by stronger hydrogen bonds, which is important for its biological activity.

### EMR Interaction With the CNS

EMR can interact with the CNS in various ways, potentially affecting chemical reactions such as radical pair formation, paramagnetic effects, DNA methylation, phosphorylation, voltage-gated channel activity, interactions with dielectric species like iron in hemoglobin and cytochromes, and GPCR mechanisms. These interactions may occur either through photon absorption or through direct or induced energy from the electromagnetic field.

The energy that an EMR carries depends mainly on its amplitude and frequency. According to electromagnetic theory, this energy can be described in two ways: as energy density (which is proportional to the square of the wave's amplitude) and as photon energy (which is proportional to the wave's frequency). The energy density (u) of EMR in free space is given by:

u=(½)ε_{O}|E|²+(½μ_{O})|B|²

where:
a. u is the energy density,
b. ε_{O} is the permittivity of free space,
c. E is the electric field strength,
d. μ_{O} is the permeability of free space,
e. B is the magnetic field strength.

In the CNS, ε and μ differ than in free space.

For non-thermal, nonionizing ELF EMR, such as those commonly found in nature, this energy is too low to break chemical bonds or induce isomerization in molecules like oxytocin. In contrast, the energy of a photon is given by:

E=h f

where:
a. E is the energy of the photon,
b. h is Planck's constant,
c. f is the frequency of the EMR. High-frequency waves (like X-rays and gamma rays) have much more energy than low-frequency waves (such as radio waves). Unlike molecules such as retinal, oxytocin does not efficiently absorb photons for isomerization.

When comparing EMR of different frequencies, higher frequencies tend to generate eddy currents that limit penetration into biological tissues, a phenomenon known as the “skin effect.” Penetration increases with the amplitude of the wave, since energy is proportional to amplitude squared. Additionally, the shape of the wave matters: square waves transmit more energy than sine waves of the same amplitude and frequency, because the rate of change (dB/dt) is greater in square waves, which are composed of multiple sine wave harmonics. Notably the chaotic activity of the CNS may be especially sensitive to perturbation from ELF EMR.

### Elf EMR and Effects on the CNS

Over the years, extensive research has explored how external ELF EMR affects the CNS, with findings often contradictory. These studies generally fall into two categories: those where EMR causes ionization or thermal effects which lead to tissue damage and those where no such destructive effects occur. High-frequency, high-energy EMR (such as gamma rays and X-rays) penetrate deeply and can cause significant injury, while ELF EMR penetrates tissues extensively but does not produce ionization or thermal damage. In contrast, visible light has limited ability to penetrate biological tissues.

Numerous studies have shown that ELF EMR in the 1-100 Hz range can produce CNS effects, such as the induction of phosphenes which are visual sensations resulting from occipital cortex stimulation without external light. Other effects of ELF EMR have also been reported. Notably, natural sources of ELF EMR, such as Schumann resonances and geomagnetic fluctuations, have been linked to psychiatric changes at these frequencies. Some research has found correlations between geomagnetic storms and increased rates of depression, anxiety, and even suicide.

Should non-human intelligent life attempt to communicate with humans, ELF EMR could provide a way to interact with the human CNS safely, without causing tissue damage, a concept that finds a parallel in the Genesis 1:3-4 passage about the creation of light. In summary, ELF EMR can penetrate the CNS without causing ionization or thermal injury and may transmit information that helps explain phenomena like neural synchrony.

### Phosphenes and Elf EMR

Phosphenes are visual sensations such as flashes, spots, or patterns of light that occur without any external light source. These sensations can be triggered by mechanical, electrical, or magnetic stimulation, most likely affecting the retina or the visual cortex. Phosphenes are the most exhaustively documented effect of ELF EMR on the CNS, making them a practical surrogate for safety assessment. Phosphenes are most reported when exposed to ELF EMR in the 10-20 Hz range and at frequencies below 100 Hz, which coincide with natural brain rhythms like alpha and theta waves.

Because ELF EMR have long wavelengths and low energy, they can pass through biological tissues, including the skull and brain, with minimal loss of strength. The effect is most noticeable when the magnetic field strength exceeds a certain threshold, typically several millitesla (mT). When a person's head is exposed to a sufficiently strong ELF EMR, weak electric currents are induced in neural tissues, including the retina and possibly the visual cortex.

The minimum magnetic field strength required to produce phosphenes varies among individuals but is generally between 5 and 10 mT at 10-20 Hz. Induced electric fields are in the order of microvolts to millivolts per meter enough to affect excitable cells but not break chemical bonds and cause isomerization. This phenomenon is a classic example of electromagnetic induction in biological tissue, resulting in a sensory experience without external light. Importantly, phosphenes are produced by direct electrical stimulation of neurons, rather than by photochemical reactions.

### Advantages and Applications of this Invention

The ability to distinguish and quantify cis and trans oxytocin isomers enables more accurate research into the neurochemical basis of empathy, trust, and cooperative behavior. This invention provides tools for:

1. Improved diagnostic assays for monitoring participants in conflict negotiations and diagnosis and treatment of neuropsychiatric conditions related to oxytocin.

2. Enhanced drug development targeting the biologically active trans isomer.

3. Reliable biomarker identification of oxytocin isomers for clinical trials and behavioral studies.

4. Standardization of oxytocin measurement protocols across laboratories.

### Potential for Drug Development

By identifying and selectively targeting the trans isomer of oxytocin, pharmaceutical formulations can be optimized for efficacy in treating disorders related to social cognition, such as autism spectrum disorder, schizophrenia, and social anxiety. Isomer-specific analogs may reduce side effects and improve therapeutic outcomes.

### Interdisciplinary Impact

This invention bridges neuroscience, analytical chemistry, and behavioral science, providing a foundation for future research into the molecular mechanisms underlying social interaction and conflict resolution. The methods described herein may also be adapted for other neuropeptides and signaling molecules exhibiting cis-trans isomerization.

### Methods for Separating and Measuring Oxytocin Isomers

Accurately distinguishing between molecular isomers is essential for understanding their biological effects is a lesson underscored by historical cases such as thalidomide. Current analytical methods often do not differentiate between the cis and trans isomers of oxytocin, even though in vitro studies suggest a typical ratio of about 10% cis to 90% trans. Additionally, oxytocin is known to degrade over time, which can further complicate measurement.

Oxytocin concentrations in biological fluids such as urine, saliva, blood, and cerebrospinal fluid (CSF) are typically in the picogram per milliliter (pg/ml) range. Standard protocols exist for extracting oxytocin from these samples, but the challenge lies in separating the isomers after extraction. This invention introduces methods for determining the relative concentrations of cis and trans oxytocin in biological samples, including:

1. Ion-mobility Mass Spectrometry (IM-MS): Separates isomers based on their collision cross-sections.

2. High-field Nuclear Magnetic Resonance (NMR): Differentiates isomers by their distinct magnetic properties.

3. Infrared Spectroscopy: Identifies isomers through their unique absorption patterns.

4. High Performance Liquid Chromatography (HPLC): Identifies isomers through their retention times

These advanced techniques enable precise quantification of each oxytocin isomer, paving the way for more accurate research and clinical applications.

### Step-by-Step Protocol: Ion Mobility Mass Spectrometry (IM-MS) for Oxytocin Isomers

1. Sample Preparation
a. Collect biological samples (e.g., blood, cerebrospinal fluid, or purified oxytocin solution).
b. Extract oxytocin using solid-phase extraction (SPE) or liquid-liquid extraction to concentrate the peptide and remove interfering substances.
c. Buffer selection: Use appropriate buffers to maintain peptide stability and minimize isomerization during handling.

2. Instrument Setup
a. Calibrate the IM-MS instrument using standard procedures and known peptide standards.
b. Set ionization parameters (e.g., electrospray ionization) optimized for peptide analysis.

3. Sample Introduction
a. Inject the prepared sample into the IM-MS system.
b. Optimize injection volume and flow rate for best sensitivity and resolution.

4. Ion Mobility Separation
a. Apply an electric field to separate ions based on their collision cross-sections (shape and size).
b. Monitor drift times: Cis and trans isomers of oxytocin will have distinct drift times due to their different conformations.

5. Mass Spectrometry Detection
a. Detect ions using the mass spectrometer.
b. Record mass-to-charge (m/z) ratios for each isomer.
c. Identify peaks corresponding to cis and trans oxytocin by comparing drift times and m/z values to synthetic standards.

6. Quantification
a. Integrate peak areas for cis and trans isomers.
b. Generate calibration curves using known concentrations of synthetic cis and trans oxytocin.
c. Calculate relative and absolute concentrations of each isomer in the sample.

7. Controls and Validation
a. Run synthetic standards of cis and trans oxytocin to confirm peak assignments and instrument performance.
b. Include blank and spiked samples to validate extraction and measurement accuracy.

8. Reporting
a. Report the ratio and concentrations of cis and trans oxytocin in the sample.
b. Document all sample preparation, instrument settings, and analysis methods for reproducibility

### Step-by-Step Protocol: NMR Analysis of Oxytocin Isomers

1. Sample Preparation
a. Obtain the mixture containing oxytocin isomers.
b. Dissolve the sample in a deuterated solvent (commonly D2O or DMSO-d6) at a suitable concentration (typically 1-10 mg/mL).
c. Filter the solution to remove particulates using a 0.22 μm syringe filter.

2. NMR Tube Loading
a. Transfer 500-600 μL of the prepared solution into a clean NMR tube.
b. Seal the tube to prevent evaporation.

3. Instrument Setup
a. Select the appropriate NMR spectrometer (400 MHz or higher is recommended for peptide analysis).
b. Tune and calibrate the instrument for the chosen nucleus (usually \hat{ }1H and \hat{ }13C).

4. Data Acquisition
a. Run a standard 1D \hat{ }1H NMR experiment to obtain the proton spectrum.
b. Acquire 2D NMR spectra (COSY, HSQC, TOCSY, NOESY) for detailed structural information and to distinguish isomers.
c. Set parameters: Number of scans, relaxation delay, and temperature (usually 25° C.).

5. Data Processing
a. Process the spectra using NMR software (e.g., TopSpin, MestReNova).
b. Phase and baseline correct the spectra.
c. Assign peaks based on chemical shifts, coupling constants, and 2D correlations.

6. Isomer Identification
a. Compare the spectra to reference spectra for known oxytocin isomers.
b. Identify unique signals or patterns that distinguish each isomer (e.g., differences in amide proton shifts, aromatic region, or side-chain environments).
c. Quantify isomer ratios using integration of resolved peaks.

7. Reporting
a. Document the protocol, instrument settings, and results.
b. Include annotated spectra highlighting distinguishing features of each isomer

### Step-by-Step Protocol: 2D IR Spectroscopy for Oxytocin Isomers

1. Sample Preparation
a. Collect biological samples (e.g., blood, CSF, or purified oxytocin solution).
b. Extract oxytocin using solid-phase extraction (SPE) or liquid-liquid extraction to concentrate the peptide and remove interfering substances.
c. Buffer and solvent selection: Use deuterated solvents (e.g., D2O) or appropriate IR-transparent buffers to minimize background absorption.

2. Instrument Setup
a. Calibrate the 2D IR spectrometer using standard procedures.
b. Set the laser pulses to target the amide-I region (typically 1600-1700 cm⁻¹), which is sensitive to peptide backbone conformation.

3. Data Acquisition
a. Apply a sequence of ultrafast IR pulses to the sample:
   1. First pulse: Excites the sample.
   2. Second pulse: Interacts with the excited state.
   3. Third pulse: Probes the system after a controlled time delay.
b. Vary the time delay between pulses to capture dynamic vibrational interactions.

4. Data Collection
a. Record the 2D IR spectra as a function of pump (excitation) and probe (detection) frequencies.
b. Generate a 2D contour map showing diagonal peaks (fundamental vibrational modes) and cross peaks (coupling between modes).

5. Data Analysis
a. Identify diagonal peaks for cis (˜1635 cm⁻¹) and trans (˜1650 cm⁻¹) oxytocin.
b. Locate cross peaks unique to each isomer (e.g., cis: (1635, 1650) cm⁻¹; trans: (1650, 1670) cm⁻¹).
c. Integrate the area under each peak to quantify the relative concentrations of cis and trans isomers.

6. Controls and Calibration
a. Run synthetic standards of cis and trans oxytocin to confirm peak assignments.
b. Calibrate quantification using known concentrations to generate standard curves.

7. Reporting
a. Report the ratio and absolute concentrations of cis and trans oxytocin in the sample.
b. Include details of sample preparation, instrument settings, and analysis methods for reproducibility.

### Step-by-Step Protocol: HPLC Separation of Oxytocin Cis/Trans Isomers

1. Sample Preparation
a. Collect biological samples (e.g., blood, cerebrospinal fluid, urine, or saliva).
b. Add protease inhibitors and acidify immediately (e.g., with trifluoroacetic acid) to stabilize oxytocin and minimize isomerization.
c. Extract oxytocin using solid-phase extraction (SPE) or liquid-liquid extraction to concentrate the peptide and remove interfering substances.
d. Reconstitute the sample in the HPLC mobile phase or a suitable buffer.

2. Column Selection
a. Choose a column:
   1. Chiral column: For direct separation of cis/trans isomers based on stereochemistry.
   2. Reverse-phase column (e.g., C18): For separation based on hydrophobicity and subtle conformational differences.

3. Mobile Phase Preparation
a. Prepare mobile phases:
   1. Aqueous phase: Water with 0.1% formic acid or trifluoroacetic acid.
   2. Organic phase: Acetonitrile or methanol with 0.1% formic acid.
b. Optimize gradient: Start with a high percentage of aqueous phase and gradually increase organic phase to elute peptides.

4. Instrument Setup
a. Set column temperature (typically 25-35° C; optimize for isomer stability).
b. Set flow rate (e.g., 0.2-0.5 mL/min for analytical columns).
c. Calibrate the HPLC system using synthetic standards of cis and trans oxytocin.

5. Sample Injection
a. Inject sample (typically 10-50 μL) into the HPLC system.

6. Chromatographic Separation
a. Run the gradient to separate isomers:
   1. Monitor retention times; cis and trans isomers will elute at different times due to their distinct conformations.
b. Collect fractions if preparative separation is desired.

7. Detection and Quantification
a. Detect eluted peptides using UV absorbance (typically at 220 nm for peptide bonds, 275 nm for tyrosine residue) or mass spectrometry (LC-MS).
b. Identify peaks corresponding to cis and trans isomers by comparing retention times to synthetic standards.
c. Integrate peak areas to quantify the relative and absolute concentrations of each isomer.

8. Validation
a. Run synthetic standards of cis and trans oxytocin to confirm peak assignments.
b. Include blank and spiked samples to validate extraction and measurement accuracy.

9. Reporting
a. Document retention times, peak areas, and concentrations of cis and trans oxytocin.
b. Include details of sample preparation, column type, mobile phase composition, gradient profile, and instrument settings for reproducibility.

Having described my invention, I claim:

1. A method for distinguishing and quantifying cis- and trans-isomers of oxytocin in a biological sample, comprising:
a. extracting oxytocin from a biological sample using solid-phase or liquid-liquid extraction;
b. stabilizing the sample to minimize degradation and isomerization;
c. analyzing the sample using at least one technique selected from ion mobility mass spectrometry, nuclear magnetic resonance, infrared spectroscopy, or high-performance liquid chromatography;
d. identifying and quantifying cis and trans isomers by comparison to synthetic standards;
e. determining concentrations of each isomer; and
f. reporting the ratio of cis to trans oxytocin as a biomarker using automated data analysis.

2. A system for monitoring oxytocin isomer ratios in relation to conflict negotiation outcomes, comprising:
a. a device configured to generate programmable extremely low frequency (ELF) electromagnetic fields at specified frequencies and field strengths;
b. a biological sample collection module for obtaining samples before and after ELF exposure;
c. an analytical subsystem for quantifying cis- and trans-oxytocin isomers using infrared spectroscopy, nuclear magnetic resonance, ion mobility mass spectrometry, or high-performance liquid chromatography;
d. a processor configured to correlate changes in oxytocin isomer ratios with measured conflict negotiation outcomes, including EEG-based synchrony, trust, empathy, or cooperative decision-making; and
e. a software interface for real-time data visualization and adjustment of ELF parameters based on negotiation progress.

3. A method for monitoring a conflict negotiation, comprising:
a. collecting and stabilizing biological samples from human subjects for oxytocin isomer analysis;
b. quantifying cis-and trans-oxytocin isomers using validated analytical protocols and automated peak integration software;
c. analyzing the relationship between oxytocin isomer ratios and decision-making progress using a computer system; and
d. reporting results as monitoring tools for progress during negotiation.
